(19) United States
(12) Patent Application Publication
(10) Pub. No.: US 2017/0296419 A1
(43) Pub. Date: Oct. 19, 2017

(54) METHODS FOR VISUAL CARE AND ENHANCEMENT FEATURING MODIFICATION OF ACCOMMODATIVE DEMAND

(71) Applicants: Dean TRAVERS, Cambridge, MA (US); Scott XIAO, Cambridge, MA (US); Alexander WENDLAND, Cambridge, MA (US)

(72) Inventors: Dean TRAVERS, Cambridge, MA (US); Scott XIAO, Cambridge, MA (US); Alexander WENDLAND, Cambridge, MA (US)

(21) Appl. No.: 15/487,846

(22) Filed: Apr. 14, 2017

Related U.S. Application Data
(60) Provisional application No. 62/377,238, filed on Aug. 19, 2016.
(60) Provisional application No. 62/323,284, filed on Apr. 15, 2016.

(51) Int. Cl. A61H 5/00; A61B 3/113; A61B 3/11; G02B 27/01; A61B 3/032

(52) U.S. Cl. CPC A61H 5/00; G02B 27/017; A61B 3/032; A61B 3/111; A61B 3/113

(57) ABSTRACT

In various embodiments, methods of treating, improving, or preventing degradation of a user's vision incorporate the display of visual content dichoptically to the left and right eye of the user while the accommodative demand placed on the left eye and/or the right eye is altered.

### RELATED APPLICATIONS

This application claims the benefit of and priority to U.S. Provisional Patent Application No. 62/323,284, filed Apr. 15, 2016, and U.S. Provisional Patent Application No. 62/377,238, filed Aug. 19, 2016, the entire disclosure of each of which is hereby incorporated herein by reference.

### TECHNICAL FIELD

In various embodiments, the present invention relates to treatment and improvement of visual function and disorders.

### BACKGROUND

Visual disorders and deficiencies affect billions worldwide, and the lack of affordable and effective treatment options is lamentable considering the prevalence and severity of the problem. These disorders and deficiencies include amblyopia, strabismus, convergence insufficiency, divergence excess, basic exophoria, convergence excess, divergence insufficiency, basic esophoria, fusional vergence dysfunction, vertical heterophoria, accommodative insufficiency, ill-sustained accommodation, accommodative infacility, accommodation paralysis, myopia (nearsightedness), hyperopia (farsightedness), other forms of ametropia, presbyopia, and visual deficiencies caused by traumatic brain injury. The effects of these disorders are diverse and lead to a reduced quality of life in which sufferers are exposed to potential educational setbacks, inhibited depth perception, an inability to perceive the world in three dimensions (3D), difficulty tracking objects in their visual space, blurred or unclear vision, difficulty with everyday activities (e.g., driving or playing catch). Without question, visual disorders and deficiencies are neglected issues that severely decrease quality of life.

Current options provided to sufferers to improve their vision typically do not address the underlying issues; rather, sufferers are most often given tools—such as glasses or contact lenses—to cover up their symptoms. However, many vision disorders should, and can only properly, be effectively treated by addressing the root problems that cause them. A clear example is glasses, which serve only as a tool to alleviate the side-effects of a visual disorder or deficiency; even worse, by using them, the user's visual ability deteriorates at an increasing rate whereby new, more powerful lenses must be acquired over time to retain clear vision. This negative cycle of visual degradation significantly increases the chances of serious ocular problems later in life as the eye over-elongates, e.g., retinal detachment, macular degeneration, and even blindness. Furthermore, many prevalent treatments that do attempt to solve, rather than mask, the problem, are deficient in relevant, important aspects. For instance, the current most prevalent treatment for amblyopia is eye patching, an option that has not changed fundamentally in hundreds of years. Not only does eye patching typically take years to address amblyopia, but it provides an ineffective treatment option, as it does not account for the binocular nature of amblyopia. The condition does not only affect one eye; rather, it is a problem that involves the neural connections and pathways of both eyes. Due to the inadequate nature of eye patching, twenty-five percent of patients treated using the method revert after treatment and lose some or all of their vision improvements. As another example, the only option currently widely suggested as a treatment for myopia (nearsightedness) and/or hyperopia (farsightedness) is laser corrective surgery. However, this surgery is expensive, uncomfortable, and does not address the root of the problem, very similarly to glasses and contact lenses.

While some techniques have been developed to address and treat some visual disorders such as amblyopia, such techniques are typically inadequate to treat many other visual disorders or to improve otherwise “normal” visual function. Thus, there is a need for systems and techniques for the treatment of a wider variety of visual disorders, particularly those related to or involving the accommodative system of the eye.

### SUMMARY

In accordance with embodiments of the present invention, the vision of a patient (or “user”) is treated via the use of a head-mounted apparatus, which may or may not have a head strap, which displays visual content to the user dichoptically via one or more display screens. The visual content displayed to the user may be of any format and/or content, including games, video content, 3D video content, still images, virtual reality operating systems and platforms (such as Google Daydream), streamed live video, or any other combination of static or dynamically rendered pixels. In various embodiments, the visual content is varied over time, and the visual content supplied to each of the user's eyes may be substantially the same or different. For example, an image or video stream that is nominally the same may be supplied to both of the user's eyes, but one or more characteristics of the visual content supplied to one or both eyes may be varied. As another example, the visual content supplied to one eye may be dimmed or brightened, varied in contrast, or partially blurred or otherwise obscured, thereby forcing use of both of the user's eyes to correctly visualize the visual content. In various embodiments, the visual content displayed to the user may be for diagnostic purposes to determine the user's ability in areas such as visual acuity, refractive error, stereopsis, contrast sensitivity, and ocular suppression.

In various embodiments of the invention, the head-mounted apparatus adjusts the accommodative demand on one or both of the user's eyes (i.e., the demand on the user's accommodative systems in order to bring an object into focus) during or instead of varying the visual content supplied thereto. As known in the art, the ability of the eye to adjust its focal length is known as accommodation. Since a nearby object is typically focused at a further distance, the eye accommodates by assuming a lens shape that has a shorter focal length. This reduction in focal length will cause more refraction of light and serve to bring the image back closer to the cornea/lens system and upon the retinal surface. Thus, for nearby objects, the ciliary muscles contract and squeeze the lens into a more convex shape. This increase in the curvature of the lens corresponds to a shorter focal length. On the other hand, a distant object is typically focused at a closer distance. The eye accommodates by assuming a lens shape that has a longer focal length. Thus, for distant objects the ciliary muscles relax and the lens returns to a flatter shape. This decrease in the curvature of the lens corresponds to a longer focal length.

In embodiments of the present invention, the apparatus incorporates two lenses (which may be internal lenses), one for each eye of the user, and the accommodative demand on one or both of the user's eyes is varied via alteration of the optical power of one or both of the lenses and/or the distance between one or both of the lenses and the display screen(s) of the apparatus. In this manner, embodiments of the present invention may be utilized to treat, mitigate, prevent negative progression of, and/or correct various types of visual disorders and disorders related to a user's visual processing system. As known in the art, the optical power of a lens, also known as the dioptric power, refractive power, focusing power, or converging power, is the degree to which the lens converges or diverges light. Optical power is measured by opticians in a unit known as a diopter, which is the reciprocal of the focal length. For example, a lens system with a focal length of 1.8 cm (0.018 m) is a 56-diopter lens, and a lens system with a focal length of 1.68 cm is a 60-diopter lens.

For example, in various embodiments, the lenses are mounted on rails or other guides, and the lenses may be moved together or independently of each other relative to their respective display screen in response to signals from a control system. In various embodiments, the lenses may be moved via one or more worm drives or other screw-based drive systems. In other embodiments, one or both of the display screens themselves may be moved while its associated lens remains stationary, or both the display screens and the lenses may be moved. In such embodiments, the display screens may also be mounted on rails or other guides and moved via one or more apparatuses such as worm drives or other screw-based drive systems. This relative movement of one or both lenses with respect to the display screens modulates the accommodative demand placed on each eye in order to keep the visual content presented on the display screens in focus. Changing the accommodative demand placed on the user's eyes serves to stimulate the user's visual accommodation in order to keep the images displayed on the display screen in focus. This may be used to train the user's visual accommodative ability, improving one or more of their eyes' power of accommodation, and the user's visual functioning as a whole.

In general, the treatment apparatus in accordance with embodiments of the present invention features a head-mounted housing that aligns the user's eyes with two lenses (which may be internal to the housing). The apparatus also includes one or two display screens upon which the user views visual content through the lenses of the apparatus. In various embodiments, the apparatus has one unitary display screen, portions of which constitute “display screens” for each of the user's eyes. For example, a divider may be disposed within the apparatus between the lenses so that each eye may only view its respective “display screen.” In other embodiments, the field of views of the lenses may also restrict each of the user's eyes to only viewing its respective display screen. In other embodiments, the apparatus features two distinct display screens, one aligned with each lens (and thus with one of the user's eyes). Regardless, the apparatus is configured to display different visual content to each of the user's eyes, as various treatment plans require.

In various embodiments, the head-mounted apparatus may be utilized in “augmented reality” scenarios in which a view of the user's surroundings is displayed on or through the display screens. For example, the apparatus may include a front-facing (i.e., facing the same direction as the user's eyes) camera that captures images of the surroundings that substantially correspond to what the user would view without wearing the apparatus. In other embodiments, the display screens are substantially transparent, and thus allow the user to view the surroundings therethrough; in such embodiments, the display screens may also be utilized to display visual content overlaid on the user's view of the surroundings.

In various embodiments, the various components of the apparatus are assembled into a unitary headset. In other embodiments, the apparatus features a housing adapted to be worn by the user and containing the lenses through which the user views the visual content. The housing may feature an attachment mechanism, e.g., a slot and/or one or more clamps and/or posts, configured to receive and secure a mobile device to the housing such that the screen (or “display”) of the mobile device is viewable through the lenses by the user. As used herein, the term “mobile device” refers to a mobile phone or tablet capable of executing locally stored applications and supporting wireless bi-directional communication and data transfer via the Internet or the public telecommunications infrastructure. Mobile devices include, for example, IPHONES (available from Apple Inc., Cupertino, Calif.), BLACKBERRIES (available from RIM, Waterloo, Ontario, Canada), or any mobile phones equipped with the ANDROID platform (available from Google Inc., Mountain View, Calif.); tablets, such as the IPAD and KINDLE FIRE; and personal digital assistants (PDAs). The bi-directional communication and data transfer can take place via, for example, one or more of cellular telecommunication, a wired connection, a Wi-Fi LAN, a point-to-point Bluetooth connection, and/or an NFC communication.

In various embodiments, the apparatus includes a control system that controls various aspects and elements of the apparatus during user treatments. The control system may include one or more processors (or “computer processors”) for controlling the various operations and elements of the apparatus, as well as a memory for storing visual content and/or instructions, conceptually illustrated as a series of modules, for controlling treatment routines and the operation of the apparatus. The control system may also include a communications interface for wired and/or wireless communication. For example, the apparatus may receive visual content from remote storage or stream visual content (e.g., from a satellite or terrestrial feed) via the communications interface for display to the user during treatment. The control system may also communicate with one or more remote databases for, e.g., storage of treatment plans and/or patient data (e.g., responses to various treatment regimens). In embodiments in which the apparatus includes and utilizes a mobile device for display of visual content, all or part of the control system may be resident within the mobile device.

In addition to or in conjunction with any of the various treatment techniques described herein, the apparatus may determine the interpupillary distance (IPD) of the user and align the lenses with the user's eyes (e.g., with the optical axes thereof). The apparatus may also align each of the lenses with the user's eyes independently without having first calculated the IPD. For instance, an eye-tracking system (described below) may provide information to the control system as to the relative alignment of the lens and the user's eyes and then the control system may appropriately adjust the lenses. For example, one or more rear-facing cameras facing the user's eyes (e.g., proximate the display screens) may be utilized to capture an image of the user's eyes, and the control system may determine the IPD based on the image and move one or both lenses laterally so that they are aligned with the user's eyes. For example, the center-to-center distance between the lenses may be substantially equal to the IPD. The rear-facing camera may be incorporated on or within the mobile device in embodiments incorporating the mobile device.

In various embodiments, the apparatus also includes an eye-tracking system for detecting and/or following the movement of the user's eyes (and therefore the direction in which each eye is looking). For example, one or more rear-facing cameras may capture still or video images of the user's pupils, and the control system may calculate the viewing direction of each pupil based on the images. (As used herein, “rear-facing” refers to facing the direction toward the user's eyes; since the display screen(s) also face the user's eyes in typical embodiments, this may be substantially the same direction. Thus, in embodiments in which mobile devices are utilized, a “rear-facing” camera will typically face the same direction as the display screen.) The delivery of the visual content may be varied by the apparatus in response to the tracking of the user's eyes. For example, the apparatus may alter the placement of visual content on the display screens depending on the degree of eye-turn, e.g., if one or both eyes are strabismic. In various embodiments, the apparatus may alter the brightness, contrast, resolution, and/or other visual quality or characteristic of the visual content based on where the user is looking.

Embodiments of the invention may dynamically manipulate and/or modify visual content presented to the user of the apparatus by one or more of (i) blurring of at least a portion of one or both of the right-eye view and the left-eye view, (ii) moving objects with respect to a fixed background, (iii) moving objects in a right-eye view with respect to a left-eye view, (iv) changing the brightness, color, and/or other visual aspects of the display background with respect to the right-eye view and/or the left-eye view or vice versa, (v) modifying the transparency value of the right-eye view and/or the left-eye view, (vi) moving the right-eye view and/or the left-eye view to accommodate different viewing axes, (vii) rapidly alternating the visibility of the left-eye view or right-eye view or one or more sub-views thereof, or (viii) separating the left-eye view and right-eye view into smaller sub-views and manipulating one or more of the smaller sub-views independently.

In various embodiments, one or more objects in the view observed by the user's left eye may be moved with respect to the view observed by the user's right eye, or vice versa, thereby stimulating the user's eyes to operate in coordination as they are intended to when operating healthily and properly.

In various embodiments of the invention, the performance exhibited by the user during use of the apparatus may be monitored and/or evaluated. The display of visual content to the user (e.g., to one or both eyes of the user) may be altered according to the evaluation of the user's performance. Reports describing the user's performance may be generated at a specific point in time or over a predetermined time interval.

In various embodiments, a synthetic background may be added to or incorporated within the visual content displayed to at least one of the user's eyes. The synthetic background may include, consist essentially of, or consist of a frequency of pink noise, white noise, brown noise, or combinations thereof. Various synthetic backgrounds may be stored and retrieved with other visual content or may be generated by the apparatus directly during display of other visual content.

While many embodiments of the invention detailed herein involve the adjustment of the accommodative demand on one or both of the user's eyes while varying the visual content supplied thereto, other embodiments display any of a variety of visual content to one or both of the user's eyes without adjusting the accommodative demand on the user's eyes during the display of visual content—such embodiments may be implemented by a head-mounted apparatus lacking the capability to adjust the accommodative demand or by a head-mounted apparatus having but not utilizing the capability.

Embodiments of the present invention may be utilized to treat or otherwise improve the vision of users having any number of visual disorders, including but not limited to amblyopia, strabismus, convergence insufficiency, divergence excess, basic exophoria, convergence excess, divergence insufficiency, basic esophoria, fusional vergence dysfunction, vertical heterophoria, accommodative insufficiency, ill-sustained accommodation, accommodative infacility, accommodation paralysis, myopia (nearsightedness), hyperopia (farsightedness), other forms of ametropia, presbyopia, and visual deficiencies caused by traumatic brain injury.

In an aspect, embodiments of the invention feature a method of treating, improving, or preventing degradation of a user's vision utilizing a treatment system. The treatment system includes, consists essentially of, or consists of a head-mounted apparatus and a control system. The head-mounted apparatus has (i) first and second display screens for, respectively, displaying visual content dichoptically to left and right eyes of the user, (ii) a first lens positioned for observation of the first display screen by the left eye, and (iii) a second lens positioned for observation of the second display screen by the right eye. The control system is configured to control (a) the visual content displayed on the first display screen, (b) the visual content displayed on the second display screen, (c) relative movement between the first and second display screens and the first and second lenses, and (d) accommodative power of the first and second lenses. Visual content is displayed dichoptically to the left and right eye of the user using the first and second display screens. During the display of visual content, the control system is used to (i) alter a distance between (a) the first lens and the first display screen and/or (b) the second lens and the second display screen, and/or (ii) alter the optical power of the first lens and/or the second lens, whereby an accommodative demand placed on at least one of the left eye or the right eye is altered.

Embodiments of the invention may include one or more of the following in any of a variety of combinations. Prior to and/or during display of the visual content, an interpupillary distance of the user may be determined. The control system may be used to align the first and second lenses with the left and right eyes, respectively, of the user based at least in part on the interpupillary distance. Determining the interpupillary distance of the user may include, consist essentially of, or consist of (i) acquiring an image of the left and right eyes with the apparatus, and (ii) with the control system, calculating a distance between pupils of the left and right eyes based on the image. Prior to and/or during display of the visual content, one or more images of the left and right eyes may be acquired with the apparatus (e.g., with one or more rear-facing cameras). The first and second lenses may be aligned with the left and right eyes, respectively, based at least in part on the one or more images. Displaying the visual content may include, consist essentially of, or consist of varying a visible characteristic of at least a portion of the visual content displayed on the first and/or second display screens. Displaying the visual content may include, consist essentially of, or consist of alternately displaying on the first and/or second display screens, (i) the visual content having the varied visible characteristic, and (ii) the visual content without the varied visible characteristic. The visible characteristic may include, consist essentially of, or consist of brightness, contrast, spatial frequency, and/or blur.

Displaying the visual content may include, consist essentially of, or consist of (i) displaying the visual content on one of the first or second display screens at a first brightness level, and (ii) displaying the visual content on the other one of the first or second display screens at a second brightness level lower than the first brightness level. Displaying the visual content may include, consist essentially of, or consist of (i) during a first time period, altering a contrast level of the visual content displayed on the first and/or second display screens from a first contrast level to a second contrast level lower than the first contrast level, and (ii) thereafter, during a second time period, altering the contrast level of the visual content displayed on the first and/or second display screens from the second contrast level to the first contrast level. Displaying the visual content may include, consist essentially of, or consist of blurring at least a portion of the visual content displayed on the first and/or second display screens. A viewing direction and/or visual axis of the left and/or right eyes may be tracked prior to and/or during display of the visual content. A blurred portion of the visual content may be aligned with the viewing direction and/or visual axis. Blurring at least a portion of the visual content may include, consist essentially of, or consist of (i) blurring only a first portion of the visual content displayed on one of the first or second display screens, and (ii) blurring only a second portion of the visual content displayed on the other one of the first or second display screens, the first and second portions of the visual content being complementary (i.e., when combined by the user's eyes into a single image, the first and second portions of the visual content do not completely overlap each other; in various embodiments, the first and second portions of the visual content do not overlap each other at all, although in other embodiments the first and second portions may overlap to some extent).

Displaying the visual content may include, consist essentially of, or consist of (A) displaying on the first display screen (i) a first version of the visual content having a first brightness level, at least a portion of the first version being blurred, and (ii) overlaid on the first version of the visual content, a second version of the visual content having a second brightness level higher than the first brightness level, the second version being unblurred, (B) displaying on the second display screen (i) a third version of the visual content having a third brightness level, at least a portion of the third version being blurred, and (ii) overlaid on the third version of the visual content, a fourth version of the visual content having a fourth brightness level higher than the third brightness level, the fourth version being unblurred, and (C) altering a transparency of the second version and/or the fourth version of the visual content. The transparency of the second version and/or the fourth version may be modulated between different levels of transparency at an alternation rate. The alternation rate may range from approximately 2 Hz to approximately 100 Hz, or from approximately 2 Hz to approximately 50 Hz. The apparatus may include at least one front-facing camera for acquiring visual content for display on the first and/or second display screens. Additional visual content may be overlaid on visual content acquired by the at least one front-facing camera. The apparatus may include (i) a housing in which the first and second lenses are disposed, (ii) a mobile device having a display, the first and second display screens being different portions of the mobile-device display, and (iii) an attachment mechanism for attaching the mobile device to the housing.

A position of visual content displayed on the first display screen and/or a position of visual content displayed on the second display screen may be altered based at least on part on a viewing direction of the left and/or right eyes. The position of the visual content may be altered based at least in part on one or more images of the left and/or right eyes. The position of the visual content may be altered based at least in part on feedback received from the user. Displaying the visual content may include, consist essentially of, or consist of, on at least one of the first or second display screens, alternating between displaying the visual content and not displaying the visual content at a predetermined frequency. Displaying the visual content may include, consist essentially of, or consist of displaying the visual content over a synthetic background displayed on the first and/or second display screens. The synthetic background may include, consist essentially of, or consist of white noise, pink noise, red noise, gray noise, and/or brown noise. Displaying the visual content may include, consist essentially of, or consist of (i) displaying the visual content on the first display screen at a position offset from a center of the first display screen in a first direction, and (ii) displaying the visual content on the second display screen at a position offset from a center of the second display screen in a second direction opposite the first direction. The positions of the visual content displayed on the first and second display screens may be dynamically adjusted such that the visual contents approach each other and/or move away from each other. Displaying the visual content may include, consist essentially of, or consist of moving the visual content across at least a portion of the first display screen and/or the second display screen over a predetermined time period. The user may be instructed to perform physical exercise before, during, and/or after display of the visual content.

In another aspect, embodiments of the invention feature a head-mounted apparatus for treating, improving, or preventing degradation of a user's vision. The apparatus includes, consists essentially of, or consists of (i) first and second display screens for, respectively, displaying visual content dichoptically to left and right eyes of the user, (ii) a first lens positioned for observation of the first display screen by the left eye, (iii) a second lens positioned for observation of the second display screen by the right eye, (iv) a housing in which the first and second lenses are disposed, and (v) a control system configured to control (a) the visual content displayed on the first display screen, (b) the visual content displayed on the second display screen, (c) relative movement between the first and second display screens and the first and second lenses, and (d) accommodative power of the first and second lenses.

Embodiments of the invention may include one or more of the following in any of a variety of combinations. The apparatus may include one or more rails on which each of the first and second lenses are mounted. The apparatus may include a drive mechanism for moving the first and second lenses relative to the first and second display screens. The apparatus may include a mobile device. The first and second display screens may be different portions of a display of the mobile device. The apparatus may include an attachment mechanism for attaching the mobile device to the housing. At least a portion of the control system may reside within the mobile device. The apparatus may include at least one front-facing camera, positioned to face away from the eyes of the user, for acquiring visual content for display on the first and/or second display screens. The apparatus may include at least one rear facing camera, positioned to face toward the eyes of the user, for acquiring images of the eyes of the user.

In yet another aspect, embodiments of the invention feature a method of treating strabismus of a user's eye utilizing a treatment system. The treatment system includes, consists essentially of, or consists of (i) a head-mounted apparatus having first and second display screens for, respectively, displaying visual content dichoptically to left and right eyes of the user, and (ii) a control system configured to control (a) the visual content displayed on the first display screen, (b) the visual content displayed on the second display screen. One or more images of one or both eyes of the user are acquired. The visual axis of one or both of the eyes is identified based at least in part on the one or more images, whereby the visual axes are not aligned with each other. Visual content is displayed dichoptically to the left and right eye of the user using the first and second display screens. Positions of visual content displayed on the first and second display screen are selected, based on the identified visual axes, such that the visual content is aligned with the visual axes of the user's eyes. Thereafter, the position of visual content displayed on the first and/or second display screens is altered such that, when the visual content is viewed by the user, the visual axes of the eyes are more closely aligned (e.g., the visual axes are more parallel and/or an angle between the visual axes is decreased and/or the visual axes more closely approach the alignment of normal, healthy eyes of a user not suffering from strabismus or other visual disorders).

Embodiments of the invention may include one or more of the following in any of a variety of combinations. The head-mounted apparatus may include a first lens positioned for observation of the first display screen by the left eye, and a second lens positioned for observation of the second display screen by the right eye. The control system may be configured to control (i) relative movement between the first and second display screens and the first and second lenses, and/or (ii) accommodative power of the first and second lenses. During display of the visual content to the user, the control system may be used to (i) alter a distance between (a) the first lens and the first display screen and/or (b) the second lens and the second display screen, and/or (ii) alter the optical power of the first lens and/or the second lens, whereby an accommodative demand placed on the left eye and/or the right eye is altered. The apparatus may include at least one front-facing camera for acquiring visual content for display on the first and/or second display screens. The apparatus may include (i) a housing in which the first and second lenses are disposed, (ii) a mobile device having a display, the first and second display screens being different portions of the mobile-device display, and (iii) an attachment mechanism for attaching the mobile device to the housing. The apparatus may include at least one rear-facing camera for acquiring the one or more images of the eyes of the user.

In another aspect, embodiments of the invention feature a method of treating a user's vergence disorder utilizing a treatment system. The treatment system includes, consists essentially of, or consists of (i) a head-mounted apparatus having first and second display screens for, respectively, displaying visual content dichoptically to left and right eyes of the user, and (ii) a control system configured to control (a) the visual content displayed on the first display screen, (b) the visual content displayed on the second display screen. Visual content is displayed dichoptically to the left and right eye of the user using the first and second display screens. During the display of visual content, the distance between the visual content displayed on the first display screen and the visual content displayed on the right display screen is altered, one or more times, to stimulate vergence movements in at least one of the eyes of the user.

Embodiments of the invention may include one or more of the following in any of a variety of combinations. The head-mounted apparatus may include a first lens positioned for observation of the first display screen by the left eye, and a second lens positioned for observation of the second display screen by the right eye. The control system may be configured to control (i) relative movement between the first and second display screens and the first and second lenses, and/or (ii) accommodative power of the first and second lenses. During display of the visual content to the user, the control system may be used to (i) alter a distance between (a) the first lens and the first display screen and/or (b) the second lens and the second display screen, and/or (ii) alter the optical power of the first lens and/or the second lens, whereby an accommodative demand placed on the left eye and/or the right eye is altered. The apparatus may include at least one front-facing camera for acquiring visual content for display on the first and/or second display screens. The apparatus may include (i) a housing in which the first and second lenses are disposed, (ii) a mobile device having a display, the first and second display screens being different portions of the mobile-device display, and (iii) an attachment mechanism for attaching the mobile device to the housing. The apparatus may include at least one rear-facing camera for acquiring the one or more images of the eyes of the user. Before and/or during the display of visual content, one or more images of the eyes of the user may be acquired. A visual axis of one or both of the eyes may be identified based at least in part on the one or more images. A display position of the visual content on the first display screen and/or the second display screen may be controlled based at least in part on the identified visual axes. For example, the position(s) on the first and/or second display screens at which visual content is displayed may be altered to align with one or both visual axes or to direct the user's visual axes toward the visual content.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean±10%, and in some embodiments, ±5%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a schematic of a head-mounted apparatus in accordance with various embodiments of the invention;

FIG. 1B depicts an exemplary view from the head-mounted apparatus in accordance with various embodiments of the invention;

FIG. 2 is a schematic of a control system in accordance with various embodiments of the invention;

FIG. 3 depicts an exemplary view from the head-mounted apparatus in accordance with various embodiments of the invention;

FIGS. 4A and 4B depict exemplary views from the head-mounted apparatus in accordance with various embodiments of the invention;

FIG. 5 depicts an exemplary view from the head-mounted apparatus featuring a synthetic background in accordance with various embodiments of the invention; and

FIGS. 6A-6C depict exemplary views from the head-mounted apparatus in accordance with various embodiments of the invention.

### DETAILED DESCRIPTION

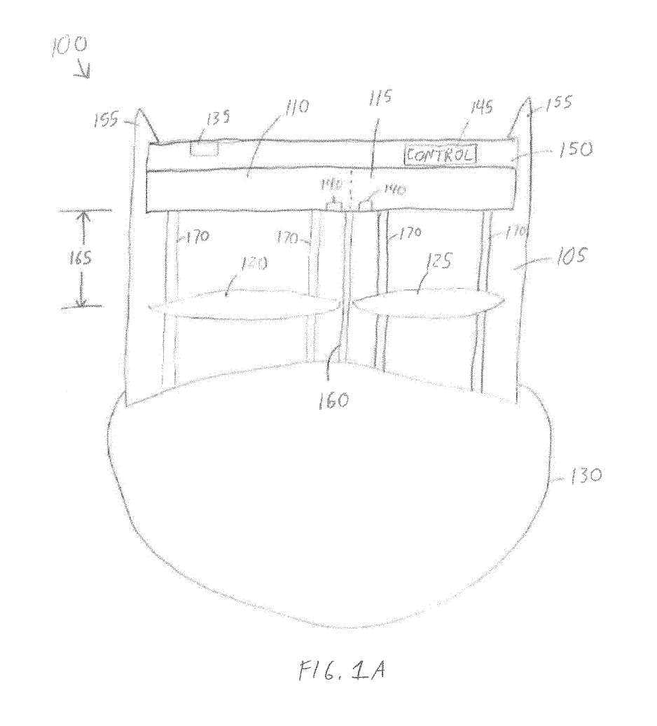

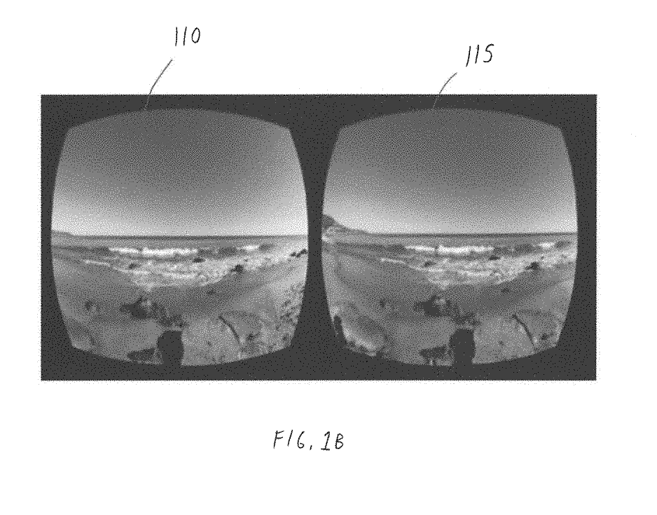

FIG. 1A is a plan view cut-away schematic of a head-mounted apparatus 100 in accordance with embodiments of the present invention. As shown, the apparatus 100 includes a housing 105 configured to be mounted on a user's head such that the user's eyes view into the housing 105. The apparatus 100 may also include display screens 110, 115 for displaying visual content to the user, as well as lenses 120, 125 each aligned with one of the display screens and through which the user views the display screens 110, 115. In order to securely mount the apparatus 100 on the head of the user, the apparatus 100 may also include one or more straps 130 and/or other fasteners.

The apparatus 100 may also include one or more front-facing cameras 135 and one or more rear-facing cameras 140. The front-facing camera 135 may be utilized to, e.g., capture visual content from the user's surroundings for display on display screens 110, 115. The rear-facing cameras 140 may be utilized to, e.g., capture still and/or video images of the user's eyes in order to measure the users IPD and/or track the eye movements of the user.

In various embodiments of the invention, display screens 110, 115 display visual content divorced from the user's surroundings, while in other embodiments, the apparatus 100 is utilized in “augmented reality” scenarios in which a view of the user's surroundings is displayed on or through the display screens 110, 115. For example, front-facing camera 135 may be utilized to capture images of the surroundings that substantially correspond to what the user would be viewing without wearing the apparatus 100. In other embodiments, the display screens 110, 115 are substantially transparent, and thus allow the user to view the surroundings therethrough; in such embodiments, the display screens 110, 115 may also be utilized to display visual content overlaid on the user's view of the surroundings. FIG. 1B is an exemplary depiction of display screens 110, 115 displaying a user's surroundings in an augmented reality mode of the apparatus 100. Such views may be overlaid with other images and/or occlusions, as detailed herein, or they may be processed (e.g., blurred) in whole or in part in accordance with embodiments of the invention.

The apparatus 100 also typically includes a control system 145 that controls the operation of the apparatus 100 and its components to facilitate treatment of the vision of the user. In various embodiments, the control system 145 is resident on the apparatus 100 (e.g., within the housing 105), while in other embodiments the control system 145 is discrete and separate from the housing and communicates therewith via wired or wireless communication. In various embodiments, the control system 145 and the display screens 110, 115 are portions of a mobile device 150, which may be receivably mounted on the housing 105. For example, the housing 105 may include one or more attachment mechanisms 155 (e.g., slots, posts, straps, clamps, etc.) adapted to receive the mobile device 150 and snugly hold it within or on the housing 105. Particularly in embodiments of the invention featuring a mobile device 150, the display screens 110, 115 may simply be portions of the display of the mobile device that may or may not partially overlap with each other. Since in various embodiments of the invention the apparatus 100 is utilized to display visual content dichoptically to the user, the apparatus may also prevent the left eye of the user from viewing the display screen 115 and the user's right eye from viewing the display screen 110. For example, the apparatus 100 may include a barrier 160 between the lenses 120, 125 that separates the fields of view of the user's eyes.

In accordance with various embodiments of the invention, the apparatus 100 may be utilized to alter the accommodative demand on one or both of the user's eyes during the display of visual content on display screens 110, 115. For example, a screen-to-lens distance 165 may be varied for one or both of the lenses 120, 125 by moving one or both lenses relative to the display screens 110, 115. Such movement may be controlled via control system 145 and may be accomplished by a variety of different means. For example, in the exemplary embodiment depicted in FIG. 1A, the lenses 120, 125 are each mounted on one or more rails 170 and may be moved thereon via, e.g., a worm drive or other drive mechanism. In various embodiments, optical power of one or both of the lenses 120, 125 is changed directly (e.g., in response to control system 145), even in the absence of relative movement between the lens and the display screen. For example, one or both of the lenses 120, 125 may include or consist essentially of electro-wetting, electronically controlled liquid crystal lenses or fluidic lenses configurable to alter the optical power of the lens; such alterations may be controlled electronically and/or via a manual actuator (e.g., a piston or screw mechanism). In one embodiment, a fluidic lens incorporates a fluid-filled membrane, and the pressure exerted on the membrane by the fluid alters the shape (e.g., curvature) of the membrane to alter the optical power of the lens. The back side of the membrane may be at least partially surrounded by one or more solid lens elements that control how the membrane deforms within the lens. The use of fluid-filled optical lenses is exemplary, and other means of altering the optical power of lenses 120, 125 may be utilized in embodiments of the invention. In “augmented reality” embodiments utilizing transparent lenses 120, 125, the optical power of such lenses may be adjusted via the use of such fluidic lenses, or the lenses may be electronically controlled liquid crystal lenses, the optical power of which may be altered by adjusting the alignment of liquid crystal molecules in the lens using a shaped electrical field (e.g., as detailed at http://lensvector.com/technology/how/ and in U.S. Pat. Nos. 8,033,054, 8,072,574, and 8,149,377, the entire disclosure of each of which is incorporated by reference herein).

In various embodiments of the invention, the optical path between the user's eyes and the display screens 110, 115 may be increased via a system of mirrors incorporated within apparatus 100. For example, the apparatus 100 may include one mirror proximate the display screens 110, 115 that faces the user's eyes and another mirror proximate the user's eyes (e.g., proximate lenses 120, 125) that faces the display screens 110, 115. In this manner, the optical path between the display screens 110, 115 may be increased (e.g., by three times or more). In various embodiments, the mirrors may be retractable into the housing 105 such that they are only visible, and only impact the optical path length, when desired. In this manner, the display screens 110, 115 may appear to be farther away from the user's eyes, facilitating the display of small objects (e.g., during visual acuity tests).

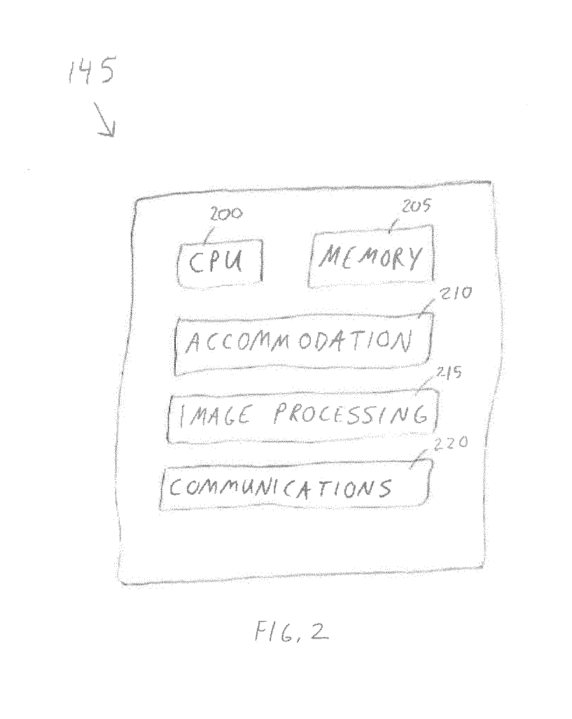

FIG. 2 is a schematic diagram of various components that may be included within control system 145 in accordance with embodiments of the present invention. As shown, the control system 145 may include a processor 200 for executing various routines and controlling the various components of the apparatus 100 as triggered within treatment procedures and/or in response to user feedback and control. The processor 200 may be one or more general-purpose processors, but in other embodiments may utilize any of a wide variety of other technologies including special-purpose hardware, programmed micro-processor, micro-controller, peripheral integrated circuit element, a CSIC (Customer Specific Integrated Circuit), ASIC (Application Specific Integrated Circuit), a logic circuit, a digital signal processor, a programmable logic device such as an FPGA (Field Programmable Gate Array), PLD (Programmable Logic Device), PLA (Programmable Logic Array), RFID processor, smart chip, or any other device or arrangement of devices that is capable of implementing the steps of the processes of the invention. The processor 200 may include or consist essentially of two or more discrete processing units, e.g., a central processing unit (CPU) and a graphics processing unit (GPU), each of which may be designed for specific tasks (e.g., rendering and processing of visual content).

The control system 145 may also incorporate local and/or remote memory 205 for, e.g., storage of treatment protocols, visual content, and/or user or treatment data. The memory 205 may include or consist essentially of one or more RAM or ROM memory chips embedded within the apparatus 100 and/or “cloud-based” remote memory accessible to apparatus 100 via wired or wireless communication. Other removable/nonremovable, volatile/nonvolatile computer storage media that may be used with apparatus 100 includes, but is not limited to, magnetic tape cassettes, flash memory cards, optical disks such as digital versatile disks, digital video tape, solid state RAM, solid state ROM, and the like. The memory 205 may be connected to the control system 145 through a removable or non-removable memory interface.

As shown in FIG. 2, the control system 145 may also include one or more modules for the execution of various treatment protocols detailed herein. For example, control system 145 may include an accommodation module 210 for controlling various components of apparatus 100, e.g., lenses 120, 125, in order to alter the accommodative demand placed on one or more of the user's eyes during display of visual content to the user. The control system 145 may also include an image processing module 215 that may alter one or more characteristics of visual content supplied to one or both of the user's eyes in accordance with treatment protocols. For example, image processing module 215 may filter still and/or video images, alter brightness and/or contrast, and/or introduce various artifacts such as blur into visual content. Image processing module 215 may also generate and/or process various types of visual content, e.g., visual content such as synthetic background images or patterns that may be layered over and/or under other visual content. Control system 145 may also include a communications module 220. In various embodiments, the communication module 220 is a conventional component (e.g., a network interface or transceiver) designed to provide communications with a network, such as the Internet and/or any other land-based or wireless telecommunications network or system. Via the communications module 220, the control system 145 may receive remote instructions and/or treatment protocols and/or stream or receive visual content from any of a variety of sources. The control system 145 may also transfer treatment records, user information, treatment times, etc. to a remote database for, e.g., tracking purposes and/or statistical analysis.

Any suitable programming language may be used to implement without undue experimentation the analytical functions described above for the control system 145. Illustratively, the programming language used may include assembly language, Ada, APL, Basic, C, C++, Objective-C, Swift, C*, COBOL, dBase, Forth, FORTRAN, Java, Modula-2, Pascal, Prolog, Python, REXX, and/or JavaScript for example. Further, it is not necessary that a single type of instruction or programming language be utilized in conjunction with the operation of embodiments of the present invention. Rather, any number of different programming languages may be utilized as is necessary or desirable.

Apparatus 100 may be utilized to treat any of a variety of visual disorders and/or to exercise or improve aspects of otherwise normal (or better than normal) vision in accordance with various embodiments of the present invention. Such treatments may involve the display of visual content to the user, via display screens 110, 115, and altering the accommodative demand on one or both of the user's eyes one or more times while the visual content is being displayed. For example, during the display of the visual content, one or both of lenses 120, 125 may be moved, thereby altering the screen-to-lens distance 165, and/or the optical power of one or both lenses 120, 125 may be varied. Treatments in accordance with embodiments of the invention may involve the display of various types and configurations of visual content, and several exemplary treatments are described herein. Treatments may last for a predetermined duration (e.g., approximately 5-20 minutes), or they may be initiated and/or terminated via user command.

In an exemplary treatment in accordance with embodiments of the invention, the brightness and/or transparency of the visual content displayed on display screen 110 and/or display screen 115 may be varied over time. In an embodiment, at times the visual content may have its alpha transparency value modulated in order to blend the video frame with the view behind the video frame in the display's view hierarchy. For example, the alpha transparency of the visual content displayed to one of the user's eyes (e.g., the user's strong, non-amblyopic eye in embodiments addressing amblyopia) is set to 15% and there is a black background (the background has an RGB color value of 0, 0, 0) behind it, so each pixel that is used to display the visual content has 15% of the color of the content's pixel and 85% black. This causes the visual content to be significantly dimmed. The alpha transparency value of the visual content displayed to the user's other eye (e.g., the user's weak, amblyopic eye in embodiments addressing amblyopia) may be set to 100% so the black background behind it is not at all visible and the visual content is displayed without any dimming.

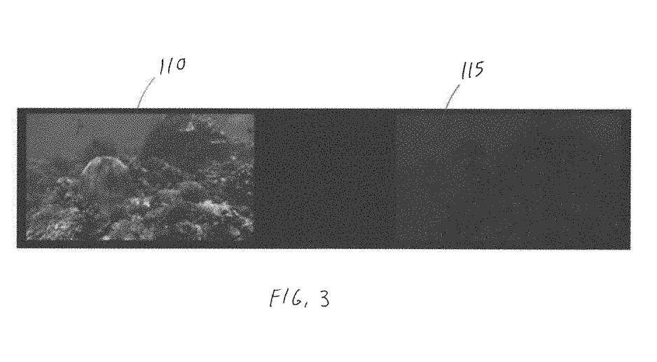

FIG. 3 depicts exemplary visual content generated by apparatus 100 in which the visual content displayed on display screens 110, 115 is substantially the same image, but the visual content displayed on display screen 115 is dimmed relative to that shown on display screen 110. For example, in the depicted embodiment, the transparency value of the visual content depicted on display screen 110 may be decreased significantly compared with that for display screen 115. In various embodiments, the dimmed visual content is displayed to the user's stronger eye, while the brighter visual content is displayed to the user's weaker eye.

Similarly, when the visual content includes images of the user's surroundings in an augmented-reality setup, the control system may dim parts or all of one or more of the display screens 110, 115. For example, as the user views the surroundings on or through the display screens 110, 115, one of the display screens (e.g., display screen 115 viewed by the right eye) may become much dimmer for reasons including but not limited to stimulating the opposite eye (e.g., the left eye) to function more significantly. It may be that the left eye's display screen (i.e., display screen 110) has a transparency value of 1.0, meaning it is fully transparent, while display screen 115 has a transparency value of 0.50, meaning it is 50% opaque. As utilized herein, “dimming” or “brightness alteration” of visual content encompasses not only modulation of the brightness of displayed visual content, but also such modulation of the transparency of a display screen through which the user observes visual content and/or the surroundings.

In accordance with embodiments of the invention, the level of transparency, brightness, and/or alpha modulation may be modified, either continuously or at intervals, for one or both eyes of the user (i.e., on one or both of display screens 110, 115). In embodiments of the present invention utilized to treat amblyopia, dimming the view of the user's fellow eye (i.e., the user's non-amblyopic eye, stronger eye, and/or the eye the user prefers to use) further stimulates the user to receive input from the amblyopic eye, which may receive a bright view with an unaltered (or brighter) transparency value; in general, it may be more comfortable for the user to view brighter visual content than it is to strain in order to see dimmer visual content. In various embodiments, the use of transparency and/or brightness modulation may be implemented on one or both of display screens 110, 115 at varying levels in order to treat a plethora of visual disorders.

In various embodiments of the invention, some of all of the visual content displayed on one or both display screens 110, 115 may be alternated between being visible (i.e., undimmed), hidden, or any value of alpha transparency in between, at rapid rates in order to stimulate visual development. The visual content displayed on display screens 110, 115 may be alternated between being visible, hidden, or any value of alpha transparency in between at different, uncorrelated rates. The rate at which the views are alternated may be entirely variable. For example, the transparency of visual content displayed on display screen 110 may be alternated at one rate (e.g., five times per second), while the transparency of visual content displayed on display screen 115 may be alternated at a different rate (e.g., 30 times per second). Not all visual content visible to each eye need be alternated at the same rate—different rates may be used for different objects visible in the visual content displayed to the same eye or both eyes. Also, as an example, if each eye is viewing multiple view elements or objects within a particular visual content, then the transparency of one or more of those elements may be rapidly alternated at a certain frequency, the transparency of one or more others of those elements may be alternated at a different frequency, and/or one or more others of the elements may remain hidden throughout.

The frequencies with which the transparency of visual content supplied to the user's eyes are alternated may not depend on each other. For example, visual content presented to the left eye may be hidden at the exact moment that the visual content presented to the right eye is not, or vice versa, or the visual content presented to both eyes may be displayed at the same time. Furthermore, the time during which the visual content has a particular transparency value may be variable. In an example embodiment, visual content may be displayed to one or both eyes for a first time period (e.g., 0.2 seconds), then rapidly hidden for a second, different time period (e.g., 0.02 seconds), and then displayed again for the first time period, or vice versa. Embodiments of the invention may utilize any of a wide variety of different time periods during which visual content is displayed and/or hidden from one or more of the user's eyes.

In another exemplary treatment in accordance with embodiments of the invention, the contrast level of the visual content displayed on display screen 110 and/or display screen 115 may be varied over time, much as brightness and/or transparency may be varied as shown in FIG. 3 and as detailed above. In one example, the contrast value of the visual content visible to both eyes (i.e., on both display screens 110, 115) is modulated from a maximum of 1.0, normal contrast, down to a minimum 0.05, low contrast, in a linear fashion across a time period of 20 seconds. The contrast level remains constant at both extremes, both the maximum and the minimum, for a time of 5 seconds before being modulated linearly once again over 20 seconds to the opposite extreme. This contrast modulation is applied to all visual content and may be combined with other exemplary treatment techniques described herein. In other embodiments, the contrast level of the visual content displayed to one eye is varied independently of the contrast level of the visual content displayed to the other eye. In yet other embodiments, the contrast level of the visual content displayed to one eye is varied while the contrast level of the visual content displayed to the other eye is held substantially constant.

Treatment procedures in accordance with embodiments of the present invention may also feature the processing of visual content to introduce artifacts (e.g., blurring, blurred regions, graphics, altered resolution, etc.) displayed to one or both of the user's eyes via display screens 110, 115. For example, the visual content supplied to one or both of the user's eyes may be processed to include a blurred region (e.g., a circular blurring) to the central area of the visual content visible to one of the user's eyes (e.g., the strong, non-amblyopic eye, in the case of a user having amblyopia). In an exemplary embodiment, the blur is a generalized 9×9 Gaussian blur positioned, at least initially, in the center of the visual content. However, the blur's position is movable with relation to the visual content; for example, when the user moves the apparatus 100 around in order to look around the video in the dichoptically presented virtual reality environment, the blur's position may be moved to remain close to the center of the user's view. Also, the blur's position may be moveable with regard to the where the user is looking, based upon the data provided by the eye-tracking performed by the apparatus in embodiments of the invention. The position of the blur when centered in the frame may have an x value of 0.5 and a y value of 0.5, indicating its center has an x value of half the total width of the frame and a y value of half the total height of the frame. In order to ensure the blur remains on the visual content, the minimum x value for the center of the blur may be greater than zero (e.g., 0.15), and the maximum x value may be less than 1.0 (e.g., 0.85). The minimum y value may be set to, e.g., 0.40, and the maximum y value may be set to, e.g., 0.60. In various embodiments, the radius of a circular blur may be set to 0.24, 24% of the total width of the video frame, and the strength of the blur may remain constant. In other embodiments, the blur strength and/or position on the display screen may vary over time.

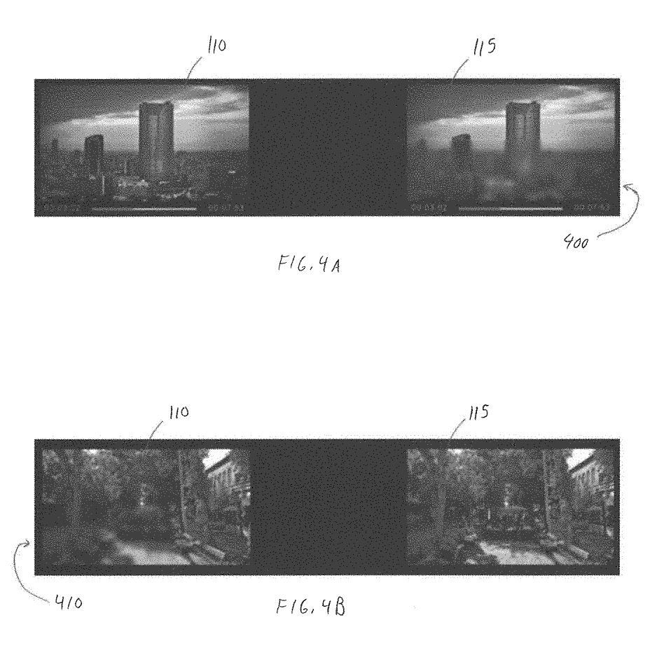

FIGS. 4A and 4B depict exemplary embodiments of visual content displayed to a user on display screens 110, 115 in which a portion of one of the images is blurred. In FIG. 4A, a blurred portion 400 of the visual content occupies (or the blur overlies) approximately the bottom half of the scene displayed on display screen 115. In FIG. 4A, a blurred portion 410 of the visual content occupies (or the blur overlies) a rectangular portion (in the bottom left quadrant) of the scene displayed on display screen 110. In various embodiments, the blur produced within the visual content is a dynamic blur, i.e., blur processed and/or modified, with multiple (or even all) of the frames of the displayed visual content. In various embodiments, one or more characteristics of the blur (e.g., the size, shape, and/or severity of blur) are modified at a predetermined (and/or variable) rate, e.g., approximately 30 times per second, although other rates may be used. The images from the visual content may be rapidly processed in order to be perceived by the user as a nearly perfect blurred copy of the unprocessed visual content. Such processing may reduce eyestrain experienced by the user, as, e.g., the color of the blurred region is approximately the same as that of the unprocessed visual content.

In various embodiments, blurring may be applied to visual content displayed on one or both of the display screens 110, 115 for the treatment of a wide array of visual disorders. In various embodiments in which the visual content includes, consists essentially of, or consists of a video stream, the blurring may not be applied directly to the video playback itself; rather, a separate view of the video stream may be generated using a blur filter, and this separate view may be displayed over the corresponding part of the original, clear video playback view. The size, position, and/or strength of the blur may be varied in accordance with embodiments of the invention.

In embodiments of the invention utilized to treat amblyopia, blurring stimulates the amblyopic eye to be used in order to see the content obscured by the blur, as the blur makes it essentially impossible for the strong eye to see the entire view alone. Such stimulation of the amblyopic eye may result in an increase in binocular vision, as both eyes begin to produce input for the brain's visual processing system. Due to neural plasticity, over time this increase in binocular vision may strengthen the correct neural pathways in order to solidify the correct usage of both eyes, thereby reducing the severity of amblyopia. However, the blur may be effectively applied to aid in the treatment of a wide array of visual disorders by stimulating and reinforcing binocular vision.

In various embodiments, the visual content may be processed to include a circular blurring to the central area of the visual content visible to one of the user's eyes (e.g., the strong, non-amblyopic eye), and to introduce a complementary blur to the outer areas of the visual content displayed to the other eye (e.g., the weak, amblyopic, eye) that are displayed unblurred to the other eye. Such embodiments ensure that, for the user to view the entire frame clearly, he or she must use both eyes in combination. The two blurs may be presented simultaneously to the user on their respective display screens.

Similarly, when the visual content includes images of the user's surroundings in an augmented-reality setup, the control system may blur parts or all of the visual content displayed on one or both of display screens 110, 115. For example, as the user views the surrounds through the display screens of apparatus 100, the central part of the display screen 110 may be blurred for various reasons including, but not limited to, stimulating the right eye to function more significantly.

In another treatment procedure in accordance with embodiments of the present invention, the visibility of the visual content delivered to one or both of the user's eyes is rapidly alternated. In such embodiments, the display screens 110, 115 may each be utilized to two sets of visual content overlaid with each other (i.e., each display screen may feature an additional “virtual screen” that overlays visual content over the visual content displayed on the physical display screen), or the apparatus 100 may include additional display screens disposed between display screens 110, 115 and lenses 120, 125. Such additional display screens may be utilized to display visual content but may also be at least partially transparent such that the visual content displayed on display screens 110, 115 behind the additional display screens is also visible to the user. In either exemplary embodiments, the “top” display screen (i.e., the screen closer to the user's eye) may display clear and unprocessed (e.g., unblurred) visual content, and the “bottom” display screen (i.e., the screen farther from the user's eye) may display highly processed versions of the visual content. For example, the processed versions of the visual content may be at least partially blurred (e.g., have a very strong Gaussian blur applied to the entire frame). In various other embodiments, the clear and unprocessed visual content may be displayed on the “bottom” display screen and the processed visual content may be displayed on the “top” display screen. In various embodiments, the visual content supplied by the two players to each of the user's eyes may be otherwise identical. The visual content of the bottom display screens may also be dimmed—e.g., this visual content may have an alpha transparency reduction applied and be positioned on top of a black background in order to dim the visual content in addition to the processed blur. The visibility of the top display screens may be rapidly alternated in order to display the heavily blurred, dimmed visual content below. This alternation may be performed at a rate of, e.g., approximately 2 Hz to approximately 50 Hz, and when the visual content supplied to one of the user's eyes is blurred and dimmed, the visual content supplied to the other eye may be substantially clear and unprocessed and vice versa. In various embodiments, the rapid alternation causes the clear visual content to be presented at a rate of 10 Hz, 100 ms, to take advantage of visually perceptible motion at a rate which resonates with the frequency required to activate the most heavily suppressed neural pathways in the visual systems of sufferers of amblyopia. Other frequencies may be used in order to activate other neural pathways.

Similarly, when the visual content includes images of the user's surroundings in an augmented-reality setup, the control system may heavily process (e.g., blur) the entirety or parts of the visual content displayed on one of the display screens 110, 115. The display screen via which one of the user's eyes observes the user's surroundings may be blurred while the other display screen remains clear, and this may be rapidly alternated eye to eye. In various embodiments, at all times one of display screens 110, 115 will be clear and the other will be blurred. The alternation may, for example, occur at a rate of, e.g., approximately 2 Hz to approximately 50 Hz. In other embodiments, at least portions of the visual content displayed on both of display screens 110, 115 are blurred, at least for particular time periods.

In another embodiment of the invention, rapid alternation of delivery of clear and processed (e.g., at least partially blurred) visual content occurs only for one of the user's eyes. In such embodiments, the visual content supplied to the other of the user's eyes (e.g., the stronger eye) may be dimmed (e.g., have an alpha transparency modulation applied to it).

In various embodiments, the movements of one or both of the user's eyes may be tracked during treatment. While the user's eyes are being tracked, the control system may be altering the nature of the treatments being displayed on the display screens. For example, the part of the visual content being viewed by the user may be the center of a blur that is dynamically applied to the visual content. That is, the treatments may or may not be adapted in order to take into account the exact location at which the user is looking. In various embodiments, treatments and/or the visual content utilized therein may be modified in accordance with the user's eye movements during the treatments. In this manner, treatment programs may be tailored to individual users without requiring direct user input from the users. For example, the visual ability of a user may be determined without asking the user for input by analyzing (e.g., via machine-intelligence algorithms) the user's eye-movement responses to visual content displayed to the user. Via such analyses, it may be determined if the user is able to converge or diverge his or her eyes appropriately to keep the visual content on the display screens in focus, determine if the user is accommodating sufficiently to keep the visual content on the display screens in focus (which may be fed back to alterations in optical power of the lenses), and/or determine if the user is looking at the appropriate location on the display screens considering the visual content (thus enabling a measure of confidence if the display of the visual content, e.g., the brightness, contrast, blurring, etc. is appropriate).

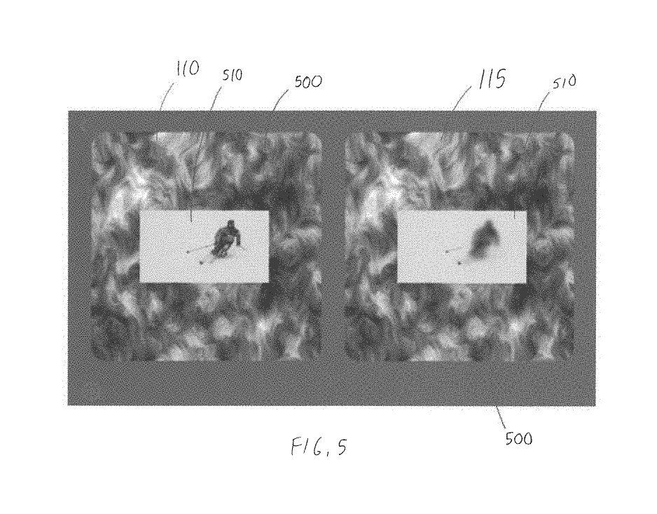

In various embodiments of the invention, the apparatus 100 may supplement or replace a portion of the visual content viewable on display screen 110 and/or display screen 115 with a synthetic background 500, as shown in FIG. 5. For example, the synthetic background may surround another portion of the visual content 510 (or even a view of the user's surroundings, in an augmented reality scenario). As shown, the portion of the visual content 510 displayed on one or both of display screens 110, 115 may be processed (e.g., blurred) in addition to being presented with a synthetic background 500, as shown for the right-eye view in FIG. 5. The synthetic background may include, consist essentially of, or consist of, for example, one or more images and/or patterns otherwise unrelated to the visual content being displayed and/or not present in the user's view of his/her surroundings (e.g., in an augmented reality scenario). In various embodiments, the synthetic background includes, consists essentially of, or consists of one or more frequencies of random noise, for example, pink noise, white noise, brown noise, red noise, gray noise, and/or combinations thereof. For example, a synthetic background including, consisting essentially of, or consisting of pink noise may create a more “quiet” background view, which may aid in the ability of the user's visual system to process or fuse visual content (e.g., images) viewed on apparatus 100. (For example, a pink noise background may make individual views on display screens 110, 115 overlap more smoothly for the user so that the chances of a potentially jarring experience for the user are minimized or reduced.) In accordance with embodiments of the invention, a synthetic background may be stored in and retrieved from memory as is other visual content, or it may be generated directly by the apparatus 100 (e.g., image processing module 215) using one or more frequency generators without undue experimentation.

Various embodiments of the invention vary the separation distance between visual content displayed on the display screens 110, 115 in order to treat a variety of different vision ailments or conditions. Specifically, the distance between the center point of visual content displayed to one eye may be moved horizontally relative to the center point between display screens 110, 115 and/or to the center point of the visual content displayed to the other eye. A common problem with an amblyopic, strabismic, or otherwise dysfunctional eye is that it does not align and track properly (i.e., it wanders and does not focus strongly on objects in the user's surroundings as it should). In order to exercise the ability of both eyes to track and align properly and in sync, various embodiments utilize moving visual content. Specifically, while the user is watching the visual content in accordance with various embodiments, the distance between the view observed by the left eye and the view observed by the right eye may be modulated. For example, this “view separation distance” may decrease to a lower limit and then increase to an upper limit. Such limits may be variable and may be set depending on the user's visual condition and ability. Furthermore, the speed with which the views converge and/or diverge may also vary.

Various embodiments may also modulate the position of the views vertically, so that the views not only converge and diverge but also move up and down. The motion of the visual content supplied to each eye need not be synchronized. For example, the left-eye view may move up as the right-eye view moves downwards. Also, the distance of vertical movement may be variable, and such motion may be combined with horizontal movement to allow for constant movement in any or all directions.

When the left-eye and right-eye views diverge, the view separation distance increases; the user's eyes are also forced to diverge in sync in order to keep the visual content in focus. This ocular divergence simulates looking at an object far in the distance. Similarly, when the left-eye and right-eye views converge, the view separation distance decreases; the user's eyes are forced to converge. This view separation distance simulates the user observing a nearby object. In various embodiments, the modulation of view separation distance forces the user's eyes to adjust and align in synchronization, thereby reinforcing the user's ability to converge and diverge their eyes and control vergence movements. This may strengthen the user's ability to track with both eyes and align them properly. The user may thus reinforce beneficial habits that promote binocular vision, and trains the user's eyes to function as a single coordinated visual system, rather than as disparate parts in a segmented visual system.

Users suffering from strabismus are unable to properly align their eyes at all times. That is, one eye may wander or always point in a direction it should not. Accordingly, various embodiments of the invention enable the alignment of the visual content displayed to each of the user's eyes so that the visual content is substantially centered along each eye's visual axis. For example, images of the user's eyes may be acquired by rear-facing camera(s) 140 to determine the direction in which each of the user's eyes is looking, and the position of the visual content displayed on display screens 110, 115 may be adjusted accordingly. In cases in which the user's eyes are not aligned with each other, the displayed position of the visual content may be modified one or more times during a treatment in order to urge the user's eyes into alignment.

In other embodiments, a user may be presented with a binocular calibration on display screens 110, 115, in which usage of both eyes is required to align the visual content properly to the visual axes of their own eyes, i.e., to the center of each eye's vision. While viewing the calibration, the user may adjust the position, in any direction, of the visual content displayed to either eye in order to align the visual content. For example, the user may control the position of the visual content displayed on one or both display screens 110, 115 via feedback supplied to the apparatus 100. In various embodiments, the calibration may be accomplished by having the user provide feedback (i.e., interacting with virtual or physical buttons/joysticks) to the apparatus 100 that indicates when the visual content is displayed in an aligned fashion. In other embodiments, the user may tilt his or her head (and thus the apparatus 100) in a particular direction and/or turn his or her head in a particular direction, and such movements (detected by, e.g., one or more gyroscopes and/or accelerometers within apparatus 100) may direct the apparatus 100 to adjust the position of displayed visual content. The relative position of the visual content at this point of alignment may be stored, e.g., in memory 205, and used to calibrate the visual position of other visual content displayed to the user in any of the embodiments of the invention detailed herein. In an exemplary calibration sequence, the two slanted lines forming an X are each displayed to a different one of the user's eyes; when properly aligned in the user's vision, the lines combine to form the X. While viewing the lines, the user may adjust the horizontal and/or vertical position of each display screen 110, 115 (i.e., the position on the screens on which the visual content is displayed) in order to have the two lines combine to form the X, thereby signaling that subsequently displayed visual content will be properly aligned to the visual axes of the user's eyes. In other embodiments, one or more other shapes or images other than an X may be utilized for calibration. As mentioned above, the positions at which the visual content is displayed to the user's eyes may be adjusted over time in order to train the user's eyes to observe the visual content in alignment with each other.

As known to those of skill in the art, when an object is presented as an image to a user's left eye, and a similar object is presented as an image to the user's right eye, the user's visual system normally attempts to fuse the two images so as to perceive one seamless, whole object. Various embodiments of the invention may present visual content either in the center of the user's field of view, or offset by some variable distance from the center of each eye's field of view. Offsetting the images may cause the user to exercise his or her ability to converge and diverge the left and right eyes in order to have the offset visual content appear as a seamless whole. For example, if the visual content presented to the user's left eye is offset to the left of the left eye's center of view, and the image presented to the user's right eye image is offset to the right of the right eye's center of view, then the user's eyes must diverge in order to see a seamless image. If the user's eyes fail to diverge, a type of double vision occurs in which two images are seen. In various embodiments, the visual content may also be offset vertically by variable distances.

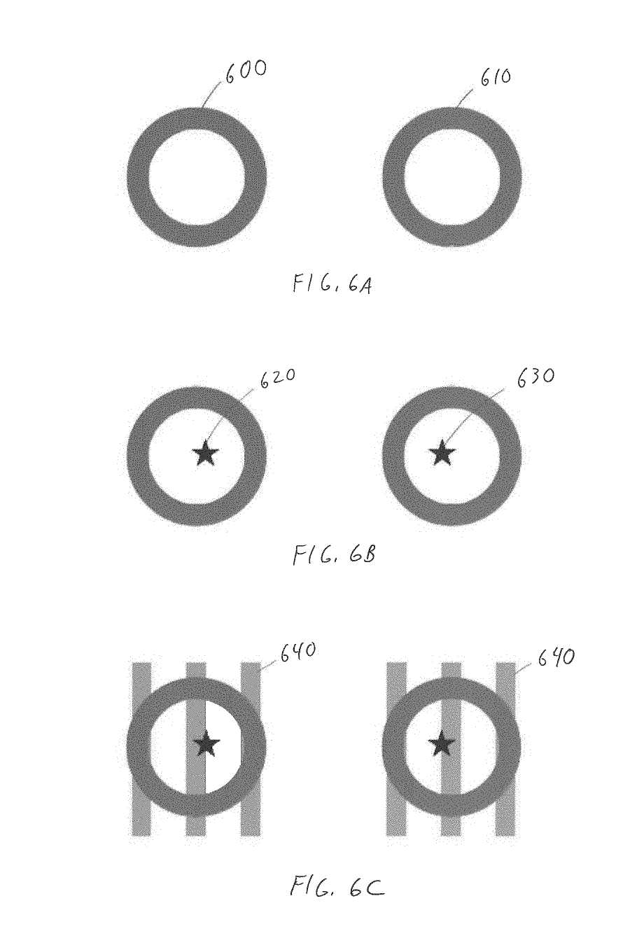

In order to further stimulate the user's visual system, embodiments of the invention may cause the user's eyes to integrate moving visual content. For example, the moving visual content (e.g., one or more objects) may diverge and converge (move outwards and inwards), thereby creating a three-dimensional (3D) effect for the user in which the visual content appears to move towards and away from them despite its position on two-dimensional display screens 110, 115. Such embodiments may strengthen the user's stereoscopic visual abilities. Users with binocular vision disorders may be benefited by exercising their ability to converge and diverge their eyes, see stereoscopic images, and use both eyes in synchronization, all of which are provided by embodiments of the invention that display such moving visual content to the user. An exemplary embodiment of is illustrated in FIGS. 6A-6C. On apparatus 100, the left circle 600 of FIG. 6A is observed on display screen 110 by the left eye of the user, and the right circle 610 is observed on display screen 115 by the right eye of the user. During exercises in accordance with embodiments of the invention, the user observes the circles 600, 610 and adjusts his or her eyes to fuse the circles such that only one seamless circle is observed.

FIG. 6B illustrates an exemplary 3D layout according to this example embodiment. The star 620 near the middle of the circle 600 is offset a predetermined distance to the right of the center of circle 600, and the star 630 near the middle of circle 610 is offset a predetermined distance to the left of the center of circle 610. When the user focuses properly on the stars 620, 630, a 3D effect is created in which a single star appears to float in front of the circle. FIG. 6C extends this exemplary embodiment via the addition of vertical bars 640 displayed to both the left and right eyes of the user. When the user focuses on the displayed visual content, the circle appears to float between the vertical bars 640 and the star. Embodiments of the invention may combine such effects with motion of one or more of the objects within the displayed visual content. For example, the stars 620, 630 may be moved horizontally in order to create the effect for the user that the combined star is moving away from and toward the user.

As known to those of skill in the art, motion perceived by a user's visual system is sufficient to trigger the firing of important visually related neural pathways. In order to stimulate such pathways, embodiments of the invention feature movement of visual content displayed on one or both of display screens 110, 115. In various embodiments, such visual content (e.g., one or more images) is rapidly moved for a variable amount of time at a variable rate of movement. The visual content may be presented to the user on one or both display screens 110, 115 while being moved in any direction for any distance across the display screen(s). For example, an image may be displayed to a user's weaker or amblyopic eye and moved across at least a portion of the display screen (e.g., approximately 5%-approximately 100% of one of the dimensions (e.g., width and/or height) of the display screen over a time period extending for less than 1 second (e.g., 0.01 second to approximately 1 second). The visual content may be moved back and forth on one or both of the display screens 110, 115 for a certain amount of time (e.g., approximately 1 second to approximately 10 seconds), at which time the visual content may be held stationary or hidden from the user's view. Such sequences may be repeated one or more times.

In various embodiments, the display of visual content and associated adjustments made by apparatus 100 may be combined with physical exercise or activities performed by the user in order to amplify the beneficial effects of the treatment. Such physical exercise or activities may occur prior to, during, or after treatment. For example, instructions for the user to exercise or perform a particular physical task may be displayed on one or both of display screens 110, 115, at which point such exercise would be performed by the user. Such exercise or activities may be of any type and/or duration. The combination of physical exercise and display of visual content may stimulate neural plasticity in the user and thereby enhance the efficacy of the treatment of the user's visual disorder.

During one or more of the treatment procedures detailed herein, the control system of apparatus 100 may at variable times independently alter the optical power of lens 120 and/or lens 125, and/or adjust the screen-to-lens distance 165 for lens 120, and/or lens 125. Such adjustments alter the accommodative demand placed on one or both of the user's eyes in order to keep the visual content displayed on display screens 110, 115 in focus. For example, the optical power of lens 120 and/or lens 125 may be altered from +2 diopters to −2 diopters and vice versa every 10 seconds in order to constantly work the user's accommodative system. In other embodiments, the optical power of one or both of the lenses may be altered between any two optical power values, and the optical power may be transitioned from one optical power to the other gradually or substantially abruptly. Such treatments may improve the power of accommodation of each of the user's eyes by exposing them to a wide array of accommodative demands in a highly controlled environment. The combination of such accommodative demand alteration with one or more of the modification treatments for visual content outlined above allow for the training of a wide array of visual abilities simultaneously.

As a further specific example, while the user is exposed to visual content displayed on the display screens of apparatus 100, the optical power of the lenses 120, 125 may be at +2, to stimulate a relaxed accommodative response to the user's eyes, as people with myopia struggle to relax the lens of their eyes properly in order to focus light on the retina at distances. Since the user is exposed to an accommodative demand at which the user struggles to focus properly, it is possible, over time, to train the user to improve his/her focusing abilities through via exposure to a wide array of accommodative demands at which the user typically struggles to accommodate.

The accommodative demand of one or both of the user's eyes may be varied during one or more treatment procedures such as those detailed herein. In other embodiments, one or more treatment procedures may be performed two or more times, and the accommodative demand on the user's eye(s) may be varied between cycles. For example, for one 15 minute treatment type the lens 120 may be set to a diopter value of +4 while the lens 125 is set to a diopter value of −4. Following this, in the next 15 minute treatment type lens 120 may be set to have a diopter value of −2 and the lens 125 may have a diopter value of +3. In yet other embodiments, the accommodative demand on one or both of the user's eyes may be varied at random intervals. In one exemplary embodiment, the control system may rapidly alternate display of processed and unprocessed visual content (as detailed above) while synchronizing the optical power of the lenses 120, 125, and thus the accommodative demand placed on the user's eyes, to coincide exactly with the alternation rate of the visual content being displayed. For example, the control system may alter the diopter power of the lens +2 for the eye when the video frame for the respective eye is blurred/dimmed and −2 for said eye when the video frame is clear (other values of optical power may also be used).

Embodiments of the invention may also involve the treatment of convergence insufficiency, other vergence disorders, or generally improving the user's vergence ability utilizing the apparatus 100. For example, visual content may be displayed to one or both of the user's eyes via display screens 110, 115. The views may be converging with regard to each other or diverging with regard to each other. That is, at any time, some or all of the visual content visible to the left eye may be moving left as some or all of the visual content visible to the right eye is moving right (diverging), or the visual content visible to the left eye may be moving right as the visual content visible to the right eye is moving left (converging). This may be done in order to alter the vergence demand on the user (i.e., the amount the user must converge or diverge in order to see a single, fused, and clear image). In order to treat convergence insufficiency embodiments of the invention train the user's ability to converge his/her eyes by presenting content that requires the user to converge.

Such embodiments of the invention may incorporate eye tracking as described herein. As the views are converging and diverging, the eye-tracking system may determine how diverged/converged the user's eyes are, and if the degree of convergence is sufficient to keep the visual content displayed on the screens fused and clear. If it is sufficient, then the control system 145 may increase the vergence demand of the visual content. If it is insufficient, then the control system 145 may decrease the vergence demand. Further, should the user suffer from strabismus, the eye tracking system may determine the degree of eye turn present in the at least one strabismic eye. Based on this determination, the visual content on the display screens may be presented in line with the visual axis of the strabismic eye. Further, over time the visual content may be presented in such a fashion as to “pull” the strabismic eye closer to alignment with the non-strabismic eye. (Such alignment of the visual axes means the orientation of the visual axes that would be exhibited by a normal person without visual deficits or disorders, as known by and recognizable to those of skill in the art; such alignment does not necessarily mean that the visual axes are parallel.) For example, should a user suffer from exotropia of 15 prism diopters (i.e., the user's eye points noticeably outwards), then the eye tracker may determine this and then present visual content two prism diopters inwards from there, forcing the user to align the strabismic eye more properly to avoid diplopia (double vision). Over time, the apparatus 100 may continually reduce the misalignment of video content in order to perfectly align the user's eyes.

Since the vergence system is intrinsically linked to the accommodative system, embodiments of the invention address both issues. For example, as the vergence demand of the visual content displayed on the display screens 110, 115 is changing, the optical power of the lenses 120, 125 may be modified by the control system 145. As an example, when the user is straining to fuse the visual content when the visual content is converging, the control system 145 may apply a +2 diopter optical power to the lenses in order to relax the user's eyes. Oppositely, the control system 145 may apply a −2 diopter optical power to the lenses in order to promote convergence. In other embodiments, the control system 145 may be altering the optical power of the lenses in a fashion that is not specifically synchronized to the alteration of the vergence demand of the visual content in order to expose the user to a wide array of accommodative demands while doing the same with vergence demands.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A method of treating, improving, or preventing degradation of a user's vision utilizing a treatment system comprising (A) a head-mounted apparatus having (i) first and second display screens for, respectively, displaying visual content dichoptically to left and right eyes of the user, (ii) a first lens positioned for observation of the first display screen by the left eye, and (iii) a second lens positioned for observation of the second display screen by the right eye, and (B) a control system configured to control (a) the visual content displayed on the first display screen, (b) the visual content displayed on the second display screen, (c) relative movement between the first and second display screens and the first and second lenses, and (d) accommodative power of the first and second lenses, the method comprising:
displaying visual content dichoptically to the left and right eye of the user using the first and second display screens; and
thereduring, using the control system to at least one of (i) alter a distance between (a) the first lens and the first display screen and/or (b) the second lens and the second display screen, or (ii) alter the optical power of the first lens and/or the second lens, whereby an accommodative demand placed on at least one of the left eye or the right eye is altered.

2. The method of claim 1, further comprising, prior to displaying the visual content:
determining an interpupillary distance of the user; and
using the control system to align the first and second lenses with the left and right eyes, respectively, of the user based at least in part on the interpupillary distance.

3. The method of claim 2, wherein determining the interpupillary distance of the user comprises (i) acquiring an image of the left and right eyes with the apparatus, and (ii) with the control system, calculating a distance between pupils of the left and right eyes based on the image.

4. The method of claim 1, further comprising, prior to displaying the visual content:
acquiring an image of the left and right eyes with the apparatus; and
aligning the first and second lenses with the left and right eyes, respectively.

5. The method of claim 1, wherein displaying the visual content comprises varying a visible characteristic of at least a portion of the visual content displayed on at least one of the first or second display screens.

6. The method of claim 5, wherein displaying the visual content comprises alternately displaying on at least one of the first or second display screens, (i) the visual content having the varied visible characteristic, and (ii) the visual content without the varied visible characteristic.

7. The method of claim 5, wherein the visible characteristic comprises at least one of brightness, contrast, spatial frequency, or blur.

8. The method of claim 1, wherein displaying the visual content comprises:
displaying the visual content on one of the first or second display screens at a first brightness level; and
displaying the visual content on the other one of the first or second display screens at a second brightness level lower than the first brightness level.

9. The method of claim 1, wherein displaying the visual content comprises:
during a first time period, altering a contrast level of the visual content displayed on at least one of the first or second display screens from a first contrast level to a second contrast level lower than the first contrast level; and
thereafter, during a second time period, altering the contrast level of the visual content displayed on at least one of the first or second display screens from the second contrast level to the first contrast level.

10. The method of claim 1, wherein displaying the visual content comprises blurring at least a portion of the visual content displayed on at least one of the first or second display screens.

11. The method of claim 10, further comprising, while displaying the visual content:
tracking a viewing direction of at least one of the left or right eyes; and
aligning the blurred portion of the visual content with the viewing direction.

12. The method of claim 10, wherein blurring at least a portion of the visual content comprises:
blurring only a first portion of the visual content displayed on one of the first or second display screens; and
blurring only a second portion of the visual content displayed on the other one of the first or second display screens, the first and second portions of the visual content being complementary.

13. The method of claim 1, wherein displaying the visual content comprises:
displaying on the first display screen (i) a first version of the visual content having a first brightness level, at least a portion of the first version being blurred, and (ii) overlaid on the first version of the visual content, a second version of the visual content having a second brightness level higher than the first brightness level, the second version being unblurred;
displaying on the second display screen (i) a third version of the visual content having a third brightness level, at least a portion of the third version being blurred, and (ii) overlaid on the third version of the visual content, a fourth version of the visual content having a fourth brightness level higher than the third brightness level, the fourth version being unblurred; and
altering a transparency of at least one of the second version or the fourth version of the visual content.

14. The method of claim 13, wherein the transparency of at least one of the second version or the fourth version is modulated between different levels of transparency at an alternation rate.

15. The method of claim 14, wherein the alternation rate ranges from approximately 2 Hz to approximately 100 Hz.

16. The method of claim 1, wherein the apparatus comprises at least one front-facing camera for acquiring visual content for display on at least one of the first or second display screens.

17. The method of claim 1, wherein the apparatus comprises:
a housing in which the first and second lenses are disposed;
a mobile device having a display, the first and second display screens being different portions of the mobile-device display; and
an attachment mechanism for attaching the mobile device to the housing.

18. The method of claim 1, further comprising altering a position of visual content displayed on the first display screen and/or a position of visual content displayed on the second display screen based at least on part on a viewing direction of at least one of the left or right eyes.

19. The method of claim 18, wherein the position of the visual content is altered based at least in part on an image of at least one of the left or right eyes.

20. The method of claim 18, wherein the position of the visual content is altered based at least in part on feedback received from the user.

21. The method of claim 1, wherein displaying the visual content comprises, on at least one of the first or second display screens, alternating between displaying the visual content and not displaying the visual content at a predetermined frequency.

22. The method of claim 1, wherein displaying the visual content comprises displaying the visual content over a synthetic background displayed on at least one of the first or second display screens.

23. The method of claim 22, wherein the synthetic background comprises at least one of white noise, pink noise, red noise, gray noise, or brown noise.

24. The method of claim 1, wherein displaying the visual content comprises:
displaying the visual content on the first display screen at a position offset from a center of the first display screen in a first direction; and
displaying the visual content on the second display screen at a position offset from a center of the second display screen in a second direction opposite the first direction.

25. The method of claim 24, further comprising dynamically adjusting the positions of the visual content displayed on the first and second display screens such that the visual contents approach each other and/or move away from each other.

26. The method of claim 1, wherein displaying the visual content comprises moving the visual content across at least a portion of the first display screen and/or the second display screen over a predetermined time period.

27. The method of claim 1, further comprising instructing the user to perform physical exercise before, during, and/or after display of the visual content.
